(19) United States
(12) Patent Application Publication
(10) Pub. No.: US 2008/0269546 A1
(43) Pub. Date: Oct. 30, 2008

(54) Self-acting urethral valve

(76) Inventors: David Wilkie, Vancouver (CA); Daniel Gelbart, Vancouver (CA)

Correspondence Address: DANIEL GELBART, 4706 DRUMMOND DR, VANCOUVER, BC, V6T-1B4, CA

(21) Appl. No.: 11/790,108

(22) Filed: Apr. 24, 2007

(51) Int. Cl. A61F 2/50

(52) U.S. Cl. 600/29

(57) ABSTRACT

The invention emulates the operation of a healthy urethra by providing a valve with a large differential between the opening pressure and the closing pressure. The differential is achieved by using a snap action created by the action of the urine flow on a force that drops off rapidly with displacement. A preferred way to achieve such as force is by using permanent magnets. The valve is installed simply by insertion into the urethra and can easily be removed. No surgical procedure is involved.

### FIELD OF THE INVENTION

The invention is in the medical field, and specifically for the prevention of urinary incontinence in men and women.

### BACKGROUND OF THE INVENTION

Incontinence is a well known medical problem becoming more common as people age. The majority of incontinence occurs in women, accelerated by anatomical changes following childbirth. Prior art treats incontinence by anatomical corrections done via minor surgery and by artificial urethral valves, restoring the ability of the urethra to act as a valve. In a healthy person the urethral muscles, acting together with the muscles contracting the bladder, are effective in achieving free flow when emptying the bladder and achieving a good seal against leakage at all other times. An ideal prosthetic urethral valve needs to open at a pre-determined point and provide as little obstruction to flow as possible. It should remain fully open as pressure drops, until bladder is completely emptied and pressure drops to a very low value. At this point valve should close and not open until original opening pressure was reached again. The problem is made more difficult by the low pressures involved. Typical opening pressure in a female should be about 20 cm of water and closing pressure should be a few cm, preferable less than 3 cm of water. Such low pressures can only supply very low forces inside the valve (a few grams) making the design of a reliable valve quite difficult. For this reason almost all prior art relies on the user to manually open and close the valve. Some prior art relies on automatic timers. The methods used by prior art to control the valve are: magnetic (bringing a strong magnet to the vicinity of the urethra to open valve), hydraulic (using a manually activated balloon or pump to inflate or deflate a cuff), electronic (wireless transmission of a control signal) and even a simple plug removed by the user in order to empty bladder. U.S. Pat. Nos. 4,457,299; 4,553,533; 4,822,333; 5,888,188; 6,183,520; 6,027,442 and 6,652,448 disclose a self acting valves however this valve do not have a sufficient difference between the opening pressure and the closing pressure, therefore starts to close before the bladder is fully voided. A more detailed explanation of the difference between a simple spring activated valve and the present invention is given in the detailed description of the invention. The fluid filled implementation disclosed in the '299 patent has even less desirable characteristics than a spring as the compression force required to collapse a sealed fluid filled enclosure goes up faster than linear. U.S. Pat. No. 5,112,306 addresses the need to prevent early closure of the valve by adding a closure delay. U.S. Pat. Nos. 5,088,980; 5,437,604; 5,722,932 and 6,213,936 use the Bernoulli effect, a liquid filled bag or a compound valve system to create some differential between the opening pressure and the closing pressure but fail to meet the requirement for very large differential and unrestricted flow, as they create the differential by severely restricting the flow. Clearly when urine pressure drops below 10 cm of water the voiding of the bladder will take a long time if the flow is very restricted, as flow rate drops with pressure. In a healthy person urination proceeds at an almost constant flow and then stops abruptly. Only a valve having a very sharp closing point set at a very low pressure (as low as a few cm of water) and minimal obstructions will emulate the operation of a healthy urethra. The prior art uses magnets for externally switching the valve between two states; the invention uses magnetic forces inside valve to achieve a self-acting valve having only two states: fully open or fully closed. It is an object of the invention to provide a simple automatic valve not requiring any external activation and having full flow as the pressure decreases during the voiding of the bladder. Another object is to provide a valve closely emulating the natural action of a healthy urethra. A further object is to provide a valve that is easy to insert and remove without need of any surgery.

### SUMMARY OF THE INVENTION

The invention emulates the operation of a healthy urethra by providing a valve having only two states, fully open or fully closed, with a large differential between the opening pressure and the closing pressure. The differential is achieved by using a snap action created by the action of the urine flow on a force that drops off rapidly with displacement. A preferred way to achieve such as force is by using permanent magnets. The valve is installed simply by insertion into the urethra and can easily be removed. No surgical procedure is involved.

### BRIEF DESCRIPTION OF THE DRAWINGS

FIG. 1 shows a cross section of the female bladder and urethra with the valve installed in the urethra.

FIG. 2 shows a graph of the flow vs. pressure of a conventional valve and of the valve according to the invention.

FIG. 3 shows a cross section of the valve as installed in the urethra.

FIG. 4 shows a cross section of an alternate embodiment.

FIG. 5 shows a version of the valve for installation on the outside of the urethra.

FIG. 6 shows the method of installing and removing the valve.

### DETAILED DESCRIPTION OF THE INVENTION

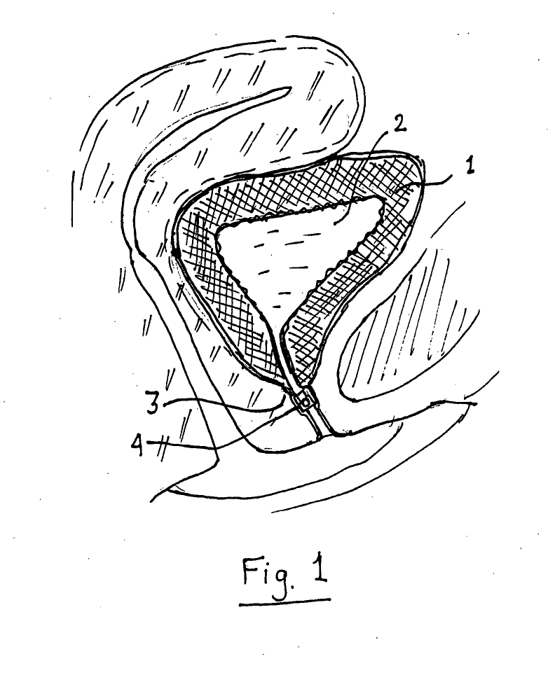

Referring to FIG. 1, valve 4 controls the flow of urine 2 from bladder 1 through urethra 3. A female anatomy is depicted but the invention is beneficial to both men and women.

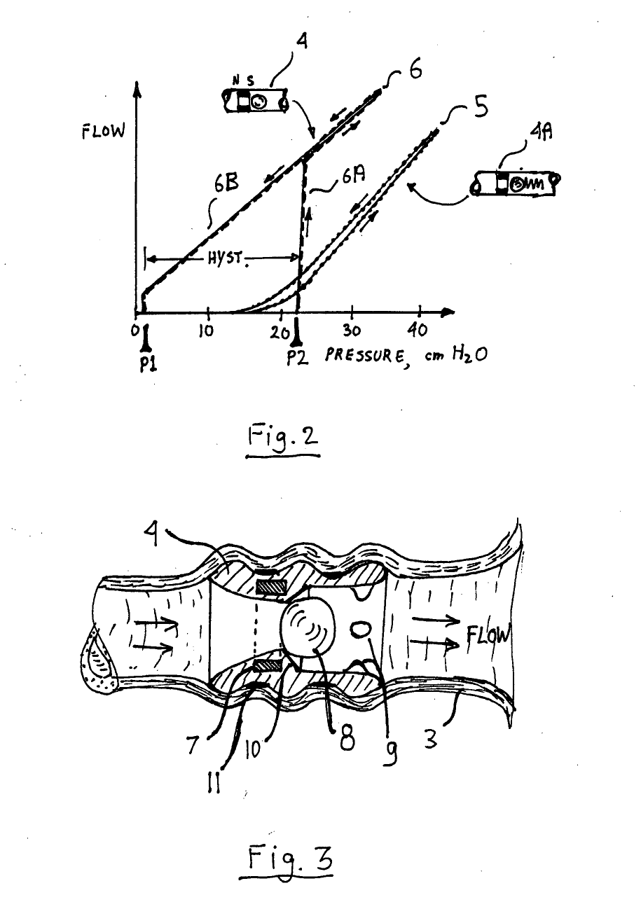

In order to emulate a correctly functioning urethra the valve needs to fully open at a set pressure and stay fully open, with as little flow restriction as possible, till bladder is empty and pressure drops to a small fraction of opening pressure. The pressure difference between the opening and closing pressure is also known as hysteresis. A simple spring loaded valve does not have the desired property. A spring provides little hysteresis, as can be seen in FIG. 2, showing flow as a function of bladder pressure. A spring loaded valve 4A will typically have a graph 5. When pressure reaches the opening point the valve opens slightly, and increased pressure increases the amount of valve opening and flow.

When pressure is reduced the graph generally traces back the increasing pressure graph. The desirable characteristic is shown in graph 6. Up to the opening pressure P2 there is no flow. At P2 the valve opens and full flow is achieved. The reason the flow keeps increasing with pressure above point P2 is because any orifice will have increased flow with increased pressure. When pressure is reduced, graph 6 resembles a graph of a simple orifice until the shut-off pressure P1 is reached. At that point the valve shuts off completely and there is no leakage. Clearly P2 needs to be significantly larger than P1 to avoid frequent urination. Valve 4 achieves the desired characteristics by the use of a magnet.

Referring now to FIG. 3, valve 4 is inserted into urethra 3 and in held in place by one or more grooves and ridges 11. The body of valve 4 is made of metal, ceramic or a polymeric material such as silicone rubber or polyurethane. A magnetic ring 7 is embedded inside valve 4 and a corrosion resistant ball made of ferromagnetic material such as type 400 stainless steel forms a valve when attracted by magnet 7 to valve body 4.

Ball 8 can be plated for increased corrosion protection. Suitable plating materials are metals such as gold or platinum or ceramic coatings. Shapes other than a solid sphere can be used for size and weight reduction. A lighter weight of ball 8 is sometimes desirable to prevent activation by inertial forces when doing vigorous exercise. Ridges 9 prevent the ball 8 from falling out but it can easily pushed in or out past ridges 9 if needed, due to the elasticity of the housing. This is desirable in case a catheter needs to be inserted into the bladder without removal of the valve from the urethra.

It is well known that the attractive force of a magnet falls off rapidly as a function of distance. The exact relationship can vary from the inverse second power of distance to as much as the seventh power, depending on the exact geometry. The ball 8 is held securely in place in housing 4 and forms a good seal, particularly with an elastomeric housing, until urine pressure separates it slightly from its seat. As soon as it separates, the magnetic force drops significantly causing the ball to be pushed by urine flow to ridges 9 and providing a large flow cross section. When urine flow stops or drops to a very low level, ball 8 is attracted back by magnet 7 and seals the valve. Since ball 8 is ferromagnetic, valve can always be activated by placing a strong magnet outside the body in the vicinity of the urethra. This will allow emptying the bladder even before full pressure was reached.

By the way of example a valve was tested with the following parameters:

Dimensions: Outside diameter 7 mm, length 10 mm, hole diameter of ball seat 4.5 mm, diameter of section housing the ball 6 mm, material is hard polyurethane (durometer of 80 Shore A).

Ball: Series 400 stainless steel ball with a diameter of 5 mm.

Magnet: Magnet ring is made of rare earth magnet with a cross section of 0.×1 mm and diameter of 6 mm. Spacing between edge of ring and ball was adjusted experimentally for opening pressure of 20 cm water. Spacing was about 0.5 mm. Magnetization was along the axis of the ring.

It was found out that the valve fully opened at 20 cm water and stayed fully open until pressure dropped to about 2 cm of water. Opening pressure could be adjusted from 10 cm water to 60 cm water by slightly changing the distance between ring and ball. These adjustments had little effect on closing pressure.

In order to prevent possible infection in the contact area between the valve and the urethra, the valve can by coated by a drug eluting coating, similar to the practice with coronary stents. A material with anti-microbial properties, such as silver, can also be used.

By the way of example, grooves 11 can have a thin silver rings at the bottom of each groove. In the alternative the complete valve can be fabricated from an anti microbial material or the polymer used to make the valve can be mixed with a material having both microbicide and fungicide properties such as zinc pyrithione or titanium dioxide. It should be understood that a suitable valve would have to be chosen for each user, based on the diameter of the urethra and the optimal opening pressure. The desired opening pressure can be established by an urodynamic test.

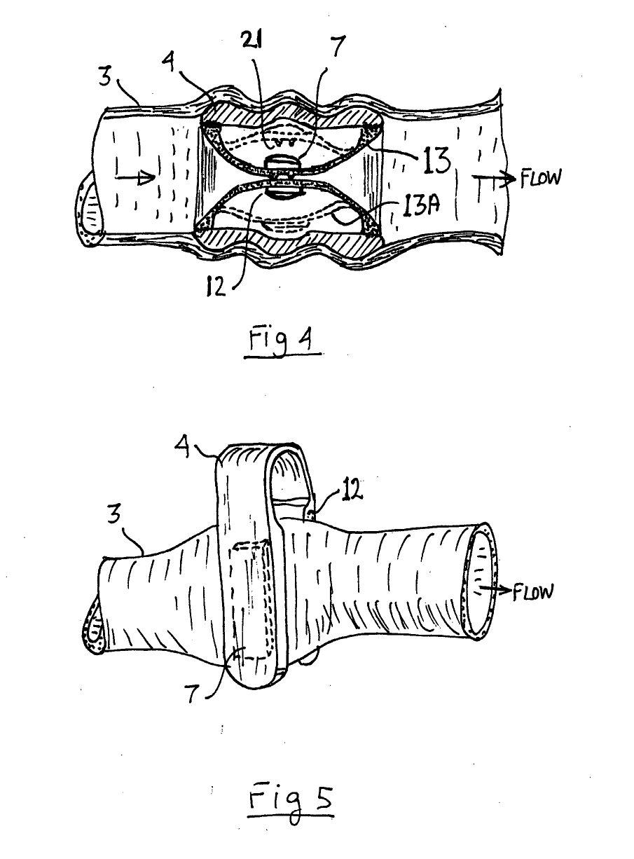

An alternate form of the valve is shown in FIG. 4. An elastomeric tube 13 is bonded at both ends to valve body 4. A magnet 7 is placed on the outside of one wall and a ferromagnetic material (or another magnet) 12 is placed on the opposing wall. When urine pressure overcomes the magnetic attraction between parts 7 and 12, the elastomeric tube snaps to position 13A. When urine pressure drops to near zero, tube snaps back to original position 13 because of the magnetic attraction between parts 7 and 12. The advantage of this configuration compared to the configuration of FIG. 3 is that it allows access to the bladder, if needed, without removing the valve. It was found out that this configuration gave less accurate set points than the one of FIG. 3. In order to reduce the required sealing pressure between the two walls of part 13, one or more fine ridges 21 can be added in the direction perpendicular to the flow. Such ridges can accommodate small particles without affecting the sealing of the valve.

A third embodiment is similar to FIG. 4 but does not use magnets. Elastomeric insert 13 is molded in a manner having two stable shapes, shown by 13 and 13A. With no pressure it snaps to shape 13, but when expanded it snaps to shape 13A until pressure drops back.

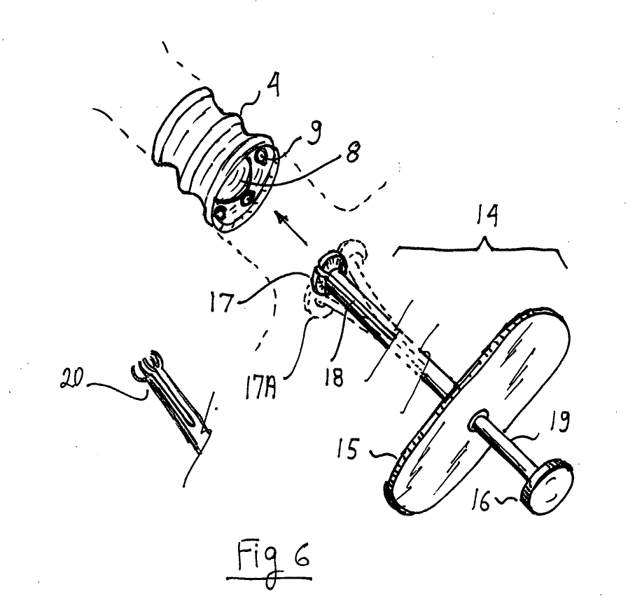

Such a snap action is well known in mechanical engineering and is used, for example, by most keyboards. It is sometimes referred to as “negative K spring”. The advantage of eliminating the metallic parts is better compatibility with MRI scanners; however, the set points were not as repeatable as the configuration of FIG. 3. Clearly elastic snap action can also be achieved by adding a separate snap action spring to the outside of tube 13. Still another configuration is shown in FIG. 5. This places the snap action outside the urethra, eliminating the risk of infection but requiring minor surgery. The principle of operation is similar to FIG. 3 and FIG. 4: a magnet 7 embedded in polymeric clip 4 is pinching-off urethra 3 by being attracted to ferromagnetic insert (or another magnet) 12 embedded in opposing part of clip 4. As in previous examples, the magnets can be replaced by a snap action spring mechanism.

FIG. 6 shows, by the way of example, a tool 14 that can be used to insert and remove valve 4. The tool has a disc-shaped tip 17 that fits into valve 4. When button 16 is pressed, tip 17 expands into the shape shown in 17A and grips valve 4. Ridges 9 prevent the valve from sliding off the expanded tool. Handle 15 is used to hold the tool similar to holding a syringe. The expansion of tip 17 is achieved by placing a longitudinal cut 18 in the tool and having the distal tip of shaft 19 elastically spread the two halves of tip 17 apart when pushed in.

The configuration of FIG. 4 allows access to the bladder without removing the valve. In the configuration shown in FIG. 3 the ball needs to be removed when access to the bladder is desired without removal of the valve. This is performed by a tool similar to tool 14 except tip 17 is shaped as to have curved fingers 20 when inserted into valve 4 fingers 20 will grip ball 8 and pull it past ridges 9. After access is no longer needed ball 8 is replaced using same tool. Expanding fingers 20 by pressing on 19 will release the ball.

What is claimed is:

1. A method for treating incontinence by installing a urethral valve having no intermediate states between being fully open and fully closed in response to urine pressure and closing at a pressure significantly below the said opening pressure.

2. A method as in claim 1 wherein difference between said opening and closing pressure is achieved by magnetic action.

3. A urethral valve having no intermediate states between being fully open and fully closed in response to urine pressure and closing at a pressure significantly below the said opening pressure

4. A valve as in claim 3 wherein said opening pressure is over 20 cm of water and said closing pressure is below 5 cm of water.

5. A valve as in claim 3 wherein difference between said opening and closing pressure is achieved by magnetic action.

6. A valve as in claim 3 wherein difference between said opening and closing pressure is achieved by snap action in an elastic material.

7. A valve as in claim 3 wherein said valve is placed inside the urethra.

8. A valve as in claim 3 wherein actuating mechanism of said valve is placed on the outside of the urethra.

9. A valve as in claim 3 wherein said valve can also be externally activated.

10. A valve as in claim 3 wherein said valve allows access to bladder without removal of the valve.

11. A valve as in claim 3 containing no metallic parts.

12. A valve as in claim 3 wherein said valve contains an anti-microbial material.

13. A valve as in claim 3 wherein said valve contains metallic silver.
